(19) United States
(12) Patent Application Publication
(10) Pub. No.: US 2014/0311259 A1
(43) Pub. Date: Oct. 23, 2014

(54) Thermal Ribbon Analysis

(71) Applicant: Quantum Signal LLC, Saline, MI (US)

(72) Inventors: David B. Johnson, Ann Arbor, MI (US); Robert M. Lupa, Vernon, IL (US); Kevin D. Melotti, Ann Arbor, MI (US); Mitchell M. Rohde, Saline, MI (US); Steven M. Rohde, Ann Arbor, MI (US); Christopher M. Showers, Royal Oak, MI (US); Michael G. Ploof, Ann Arbor, MI (US)

(21) Appl. No.: 14/183,239

(22) Filed: Feb. 18, 2014

Related U.S. Application Data
(60) Provisional application No. 61/766,104, filed on Feb. 18, 2013.

(51) Int. Cl. G01N 33/00

(52) U.S. Cl. CPC G01N 33/00
USPC 73/865.8

(57) ABSTRACT

Thermal ribbon analysis is achieved via various approaches.

### RELATED APPLICATIONS

The present patent application claims priority to the previously filed and presently pending provisional patent application of the same title, filed on Feb. 18, 2013, and assigned patent application Ser. No. 61/766,104, which is hereby incorporated by reference.

### BACKGROUND

Visual odometry is the process of estimating or measuring the motion of a vehicle through analysis of image sequences collected by a camera mounted on or of the vehicle.

### DETAILED DESCRIPTION

Thermal ribbon analysis is a forensic-focused desktop device that permits fast, efficient, and automated recovery of data on thermal mass transfer or dye diffusion thermal transfer ribbons.

We claim:

1. A thermal ribbon analysis approach.

2. A system and method for thermal ribbon analysis.
